(19) United States
(12) Patent Application Publication
(10) Pub. No.: US 2013/0274591 A1
(43) Pub. Date: Oct. 17, 2013

(54) SINGLE CHANNEL MRI GUIDEWIRE

(71) Applicants: Merdim Sonmez, Rockville, MD (US); Ozgur Kocaturk, Rockville, MD (US); Christina E. Saikus, Bethesda, MD (US)

(72) Inventors: Merdim Sonmez, Rockville, MD (US); Ozgur Kocaturk, Rockville, MD (US); Christina E. Saikus, Bethesda, MD (US)

(73) Assignee: THE UNITED STATES OF AMERICA, as represented by THE SECRETARY, DEPARTMENT OF HEALTH AND HUMAN SERV, Rockville, MD (US)

(21) Appl. No.: 13/977,557

(22) PCT Filed: Jan. 4, 2012
(86) PCT No.: PCT/US2012/020139

§ 371 Date: Jun. 28, 2013

Related U.S. Application Data
(60) Provisional application No. 61/429,833, filed on Jan. 5, 2011.

(51) Int. Cl. A61B 5/00; A61B 5/01; A61B 5/055

(52) U.S. Cl. CPC A61B 5/6851; A61B 5/055; A61B 5/01
USPC 600/411; 600/423

(57) ABSTRACT

The present application discloses a guidewire for magnetic resonance imaging with a single channel design to reduce complexity and to provide conspicuous tip visibility under MRI. In one embodiment, a guidewire body includes an antenna formed from a rod and a helical coil coupled together. The helical coil can have multiple windings without any gaps between the windings. The rod passes through the windings of the helical coil and is coupled to the helical coil using a conductive joint positioned at an end of the rod and at an end of the helical coil. Insulation can be positioned between the rod and the windings of the helical coil. The configuration allows visibility of the antenna along the length of a rod, except where it enters the windings of the coil. Thus, the tip visibility is enhanced as being separated from the rod.

### PRIORITY CLAIM

This application claims the benefit of U.S. Provisional Application No. 61/429,833, filed Jan. 5, 2011, which is incorporated herein by reference in its entirety.

### FIELD

The present application relates to interventional guidewires, and, more particularly, to such guidewires for use with interventional magnetic resonance imaging.

### BACKGROUND

Interventional Magnetic Resonance Imaging (“iMRI”) has increased in interest during the last decade due to Magnetic Resonance (“MR”) compatible instruments, the development of rapid imaging techniques and automatic instrument tracking techniques.

For MR guidance of vascular interventions to be safe, the interventionalist must be able to visualize the tip location and distal shaft of the MRI compatible guidewire relative to the vascular system and surrounding anatomy. A number of instrument visualization approaches under MRI have been developed including both passive and active techniques. Passive visualization techniques rely on the creation of susceptibility artifacts to enhance the device (e.g., catheter) appearance by using contrast agents or ferromagnetic materials. Active visualization relies on supplemental hardware embedded into a catheter body, such as a Radio Frequency (“RF”) antenna to receive the RF signal during MRI (Susil R C, Yeung C J, Atalar E, “Intravascular extended sensitivity (IVES) MR imaging antennas.” Magnetic Resonance in Medicine, 2003; 50(2): 383-390). However, none of these techniques provides satisfactory results in terms of both instrument tip and shaft visualization at the same time. Visualization of the shaft only is not enough to advance a guidewire through tortuous vessels due to the risk of puncturing vessel walls and visualization of a single point is not sufficient for steering an active guidewire in complex vessel territory (Atalar E, Kraitchman D L, Carkhuff B, Lesho J, Ocali O, Solaiyappan M, Guttman M A, Charles H K, Jr. Catheter-tracking FOV MR Fluoroscopy. Magnetic Resonance in Medicine 1998; 40(6):865-872). Patent publication number WO 2009/088936 provides the ability to visualize the shaft separately from the tip, but it requires separate channels for both the tip and the shaft, which is costly and more difficult to manufacture.

### Interventional MR Imaging

Magnetic Resonance Imaging (MRI) is one of the most important clinical imaging modalities. A significant advantage of using MRI in clinical procedures is that imaging via MR is conducted only using a strong homogenous magnetic field and radio frequency energy pulses, without the use of harmful ionizing radiation, such as with the use of X-ray angiography. Also, MRI utilizes Nuclear Magnetic Resonance principles with gradient coil elements to provide spatial encoding, resulting in the ability to perform 3-D human body imaging with high soft tissue contrast (Lauterbur P C. NMR Imaging in Biomedicine. Cell Biophysics 1986; 9(1-2): 211-214; Lai C M, Lauterbur P C. True Three-Dimensional Image Reconstruction by Nuclear Magnetic Resonance Zeugmatography. Physics in Medicine and Biology 1981; 26(5):851-856; Kramer D M, Schneider J s, Rudin A M, Lauterbur P C. True Three-Dimensional Nuclear Magnetic Resonance Zeugmatographic Images of a Human Brain. Neuroradiology 981;21(5):239-244). MRI allows one to obtain information about various physical parameters such as flow, motion, magnetic susceptibility and temperature (Axel L. Blood Flow Effects in Magnetic Resonance Imaging. Magnetic resonance Annual 1986;237-244; Henkelman R M, Stains/ G J, Graham S J. Magnetization Transfer in MRI: A review. NMR Biomedicine 2001; 14(2):57-64; Dickenson R J, Hall A S, Hind A J, Young I R. Measurement of Changes in Tissue Temperature using MR Imaging. Journal of Computer Assisted Tomography 1986;10(3):468-472). Because of this, MRI has a wide variety of both diagnostic and therapeutic imaging applications both in the clinical and research environment. When MRI was initially introduced in the clinical environment, it was used for only diagnostic imaging purposes with almost no consideration for use in therapeutic procedures (Webb W R, Gamsu G, Stark D D, Moon K L, Jr., Moore E H. Evaluation of Magnetic Resonance Sequences in Imaging Mediastinal Tumors. American Journal of Roentgenology 1984; 143(4):723-727; Belli P, Romani M, Magistrelli A, Masetti R, Pastore G, Costantini M. Diagnostic Imaging of Breast Implants: Role of MRI. Rays 2002; 27(4):259-277). Reasons for this can be attributed to the lack of sequences designed for interventional MRI such as sequences for real-time device tracking, sequences that provide image contrast that correlate directly to therapy performance, high-speed sequences that allow real-time imaging with sufficient contrast and resolution and the lack of dedicated and optimized hardware for interventional applications.

In recent years, efforts have been made to develop MRI as an interventional tool for image guided interventions by addressing the above mentioned challenges (Miles K. Diagnostic and Therapeutic Impact of MRI. Clinical Radiology 2002; 57(3):231-232; Jolesz F A, Blumenfeld S M. Interventional Use of Magnetic Resonance Imaging. Magnetic Resonance Quarterly 1994; 10(2):85-96; Jolesz FA. Interventional and Intraoperative MRI: A General Overview of the Field. Journal of Magnetic Resonance Imaging 1998; 8(1):3-7). Also, development of 1.5 T magnets with short bores that allow access to the groin area for catheter-based procedures, liquid crystal image displays that can be exposed to high magnetic fields, improvements in the hardware of the magnetic field gradient systems for additional gains in image acquisition speed, and the development of catheter based MRI antennas for localized intravascular signal reception, provide wide range of interventional MR Imaging applications.

### MRI Compatible and Visible Devices for Interventional Procedures

MR guided interventions should be performed with devices free of ferromagnetic components, otherwise as one would encounter severe magnetic forces (induced displacement force and torque) on the device by the static magnetic field of the MR scanner and they would also cause image distortion due to the intrinsic susceptibility artifact (Shunk K A, Iima J A, Heldman A W Transesophageal magnetic resonant imaging. Magn Reson. Med 1999;41:722-726). However, MR compatible and safe devices are not enough to perform vascular interventions with MRI. The reliable visualization of these devices in relation to the surrounding tissue morphology is also required. In contrast to ultrasound, X-ray fluoroscopy, or computed tomography (CT), visualization of interventional instruments in MR has proven to be difficult. A number of approaches have been developed for depicting vascular instruments in an MR environment. They can be broadly grouped into two categories: passive and active visualization.

### Passive Visualization

In passive visualization techniques, achieving adequate catheter contrast is based on enhancing the inherent signal void of an instrument as it displaces (spins) during insertion. Differences in magnetic susceptibility can be used to create large local losses in signal due to intra-voxel dephasing (Rubin D L, Ratner A V, Young S W. Magnetic susceptibility effects and their application in the development of new ferromagnetic catheters for magnetic resonance imaging. Invest radiol. 1990;25:1325-1332). The tip or body of passive catheters is composed of either ferromagnetic or paramagnetic sleeves that produce susceptibility artifacts. Incorporating multiple rings of paramagnetic dysprosium oxide (Dy2O3) along the instrument tip allows the catheter to be consistently visualized independently of orientation (Bakker C J, Hoogeveen R M, Hurtak W F, van Vaals J J, Viergever M A, Mali W P. MR-guided endovascular interventions: susceptibility based catheter and near real time imaging technique).

Susceptibility markers should have a high magnetic moment to induce an adequate artifact at a variety of scan techniques and tracking speeds. In other words, they must have sufficient contrast to noise ratio (CNR) with respect to the background in order to distinguish the device in thick slab images.

The advantage of using a passive marker is that circuit components and transmission lines are not required to visualize the catheter. This property of passive visualization techniques is important because it also eliminates electrical safety issues. However, this technique also has several disadvantages. First, it provides low spatial resolution. Second, it slows down the speed of the procedure compared to active tracking methods. And finally, a susceptibility artifact varies based on device orientation and magnetic field strength.

### Active Visualization

Active visualization relies on the incorporation of a miniature solenoid coil into the device itself (Dumoulin C L, Souza S p, Darrow R D. Real-time position monitoring of invasive devices using magnetic resonance. Magn Reson. Med. 1993;29:411-415; Ladd M E, Zimmerman G G, Mcklnnon G C, von Schulthess G K et al. Visualization of vascular guidewires using MR tracking. J Magn Reson Imaging 1998;8:251-253; Leung D A, debatin J F, Wildermuth S, McKinnon G C et al. Intravascular MR Tracking catheter: preliminary experimental evaluation. Am J roentgenol 1995; 164: 1265-1270). The coil is connected to the scanner via a transmission line such as a thin coaxial cable passing through the catheter and provides a robust signal, identifying the instrument location with high contrast. The tip of an active catheter can be visualized with high contrast by the incorporated coil on the tip.

### Loop Antenna: Solenoid Coil

A solenoid coil is basic form of loop antenna element in which the wire is coiled in a helical pattern to create a cylindrical shape. Solenoid micro coils can be connected to the MR systems through the use of coaxial or twisted pair transmission lines, which may serve both detuning and signal transduction purposes. Loop antenna signal sensitivity for small-loop receivers falls off very rapidly (l/r³, where r is the radial distance from the loop) (Balanis C A. Antenna theory. New York: John Wiley & Sons; 1997. p. 941). To improve longitudinal coverage, long-loop intravascular antennas were subsequently investigated (Atalar E, Bottomley P A, Ocali O, Correia L C, Kelemen M D, Lima J A, Zerhouni E A. High resolution intravascular MRI and MRS by using a catheter receiver coil. Magn Reson Med 1996;36:596-605). For these long, narrow loop receivers (in which the loop length is much greater than its width), sensitivity falls off as l/r².

### Loop Antenna: Opposed Solenoid Coil

The opposed solenoid loop antenna is based on groups of helical loops separated by a gap region, with current driven in opposite directions in the helical loops on either side of the gap. The gap provides the small area of homogenous longitudinal magnetic field that makes it a good candidate for especially using as an imaging coil within and beyond the vessel wall. However, it has a small area of homogenous longitudinal coverage compared with the dipole antenna.

### Dipole Antenna

A dipole antenna for iMRI applications can be a simple coaxial transmission line with an extended inner conductor. Dipole antenna sensitivity falls off as l/r where r is the radial distance from the antenna center (Susil R C, Yeung C J, Atalar E, “Intravascular extended sensitivity (IVES) MR imaging antennas.” Magnetic Resonance in Medicine, 2003; 50(2): 383-390).

Dipole antenna sensitivity can be improved by increasing the insulation layer (insulation broadens the SNR distribution) and helical winding over the extended core inductor (winding allows for improved SNR near the tip of the antenna).

### SUMMARY

The present application discloses a guidewire for magnetic resonance imaging with a single channel design to reduce complexity, while maintaining conspicuous both tip and shaft visibility under MRI.

In one embodiment, a guidewire body includes an antenna formed from a MRI compatible metal rod and a helical coil coupled together. The helical coil can have multiple windings without a gap between the windings. The rod passes through the windings of the helical coil and is coupled to the helical coil using a conductive joint. The conductive joint can be at a distal end, a proximal end, or both ends of the helical coil. When at a distal end, the conductive joint forms a conductive tip of the guidewire. Insulation can be positioned between the rod and the windings of the helical coil. The configuration allows visibility of the antenna along the shaft of the rod, but signals are suppressed where the rod passes through the coil. Thus, the tip visibility is enhanced because the suppressed signals between the tip and the shaft of the rod create a gap between the two. The gap increases visibility as it is easier to distinguish the distal tip from the rest of the shaft profile.

In another embodiment, the conductive joint is a solder joint with a semispherical shape in order to maximize conductive surface area and increase the tip visibility.

In yet another embodiment, the rod diameter can be reduced as the rod enters the windings in order to increase room for additional insulation. The additional insulation further reduces signal reception by the rod in the area of the windings.

According to one embodiment, a guidewire for use with magnetic resonance imaging comprises an antenna formed from a combination of a rod and a helical coil. The coil defines an internal space, and the rod is positioned to extend axially through the internal space and is coupled to the coil using a conductive joint at an end of the rod. The conductive joint forms a tip of the guidewire. The guidewire has a null zone defined over an axial length between the conductive joint and a point proximal of the conductive joint. The null zone is operable to suppress signals received by a portion of the rod within the null zone, thereby producing a conspicuous distal tip signal.

The null zone can produce a spatial separation between the distal tip signal and the shaft signal. The coil can have windings that are adjacent to each other over an axial distance corresponding to at least the length of the null zone.

The guidewire can comprise a temperature sensor positioned in and axially movable relative to the guidewire. The temperature sensor can be configurable to monitor in real-time temperatures of interest along the guidewire. The temperature sensor can be configurable to measure for heating increases caused by defects in the guidewire. The guidewire can comprise a dedicated port formed in the guidewire into which a distal end of the temperature sensor is inserted.

A distal end portion of the guidewire can be curved, and the helical coil can have a corresponding preformed curved configuration without gaps between adjacent windings. The helical coil can be preformed of a shape memory alloy into the curved configuration.

The guidewire can comprise insulation in an annular region between the helical coil and the rod. The guidewire can comprise multiple layers of insulation separating the rod from the coil.

The foregoing and other objects, features, and advantages of the invention will become more apparent from the following detailed description, which proceeds with reference to the accompanying figures.

### BRIEF DESCRIPTION OF THE DRAWINGS

FIG. 1 shows a distal end of a guidewire according to one embodiment.

FIG. 2 shows a more detailed view of a distal end of a guidewire according to a second embodiment.

FIG. 3 shows an extended view of a guidewire with a connector at a proximal end for connecting to a processing system.

FIG. 4 is an illustration showing the guidewire with a conspicuous tip being visible.

FIG. 5 shows another embodiment of the guidewire with a temperature sensor embedded therein.

FIG. 6 shows a cross-sectional view of the guidewire taken along lines 6-6 of FIG. 5.

FIG. 7 is a flowchart of a method that can be used to suppress portions of the guidewire in order to increase visibility of the tip.

FIG. 8 is a schematic illustration of an interventional magnetic resonance imaging system that can use the guidewire described herein.

FIG. 9A is an illustration showing a curved guidewire with a conspicuous tip.

FIG. 9B is an illustration showing, on the left side, the guidewire as shown in FIG. 4 with a conspicuous tip indicated by the horizontal line, and on the right side, a conventional guidewire showing a lack of a conspicuous tip.

FIG. 10 is a graph of normalized heating vs. time for a guidewire according to one of the embodiments.

FIG. 11 is a graph of temperature vs. time for a guidewire according to one of the described embodiments, showing that there is no overheating problem.

FIG. 12 is a magnified portion of the graph of FIG. 11.

FIG. 13 is another magnified portion of the graph of FIG. 11.

FIG. 14 is a graph comparing the magnitudes of guidewire heating in one of the described embodiments over three different test conditions.

FIG. 15 is a chart summarizing the magnitude of guidewire heating for two different embodiments over three different test conditions.

### DETAILED DESCRIPTION

The current invention relates to the iMRI guidewires in which an antenna embedded into guidewire body is used for signal reception. A receiving antenna is positioned within the imaging volume that is used to detect the MR signal generated from the patient as the excited spins relax back into an equilibrium distribution. Embodiments described herein can be used for a clinical grade 0.035″ multi purpose guidewire that can offer both precise tip location and distal shaft visualization.

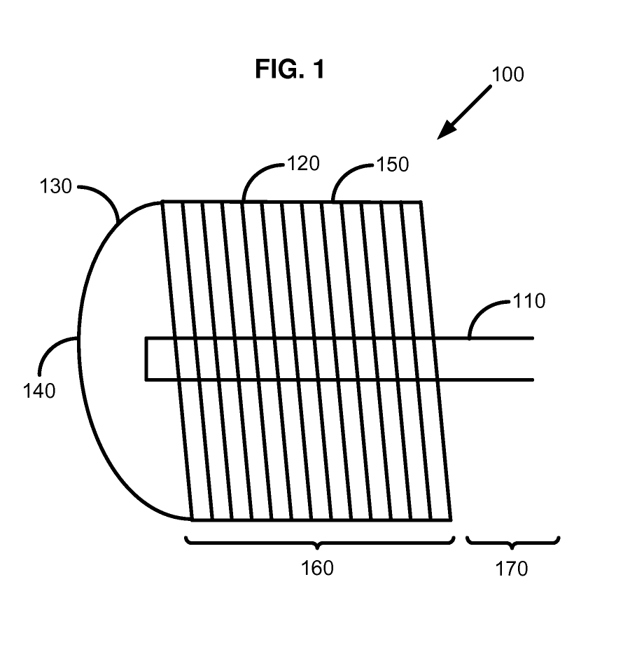

FIG. 1 shows a distal end of a guidewire 100 according to one embodiment including a single antenna formed from a rod 110 coupled to a helical coil 120 by a conductive joint 130. The rod 110 passes through the center of the coil 120 and couples to the coil at the tip 140 of the guidewire by the conductive joint. The conductive joint can be a solder joint or other means of coupling the rod to the coil. Soldering filler materials are available in many different alloys for differing applications. Examples include a eutectic alloy of 63% tin and 37% lead (or 60/40, which is almost identical in performance to the eutectic). Other alloys can also be used. The conductive joint 130 can have a semispherical shape so as to maximize a surface area to increase the signal intensity thereof. Other shapes can also be used. However, a large surface area can receive MR signals, which assists in making the tip 140 conspicuous on any resulting image. The coil 120 can be formed by a plurality of individual windings, such as winding 150, that can be tightly packed so that no gaps exist between the windings. By making the windings tightly packed, the received signal is significantly reduced for a portion of the rod 160 within the windings does not receive MR signals, while a shaft portion 170 of the rod outside of the windings does receive MR signals. This allows the tip 140 to be visible and separated from the shaft 170 by the suppressed area 160. As described further below, insulation can be placed between the coil 120 and the rod 110 to further ensure that the area 160 has suppressed MR signals.

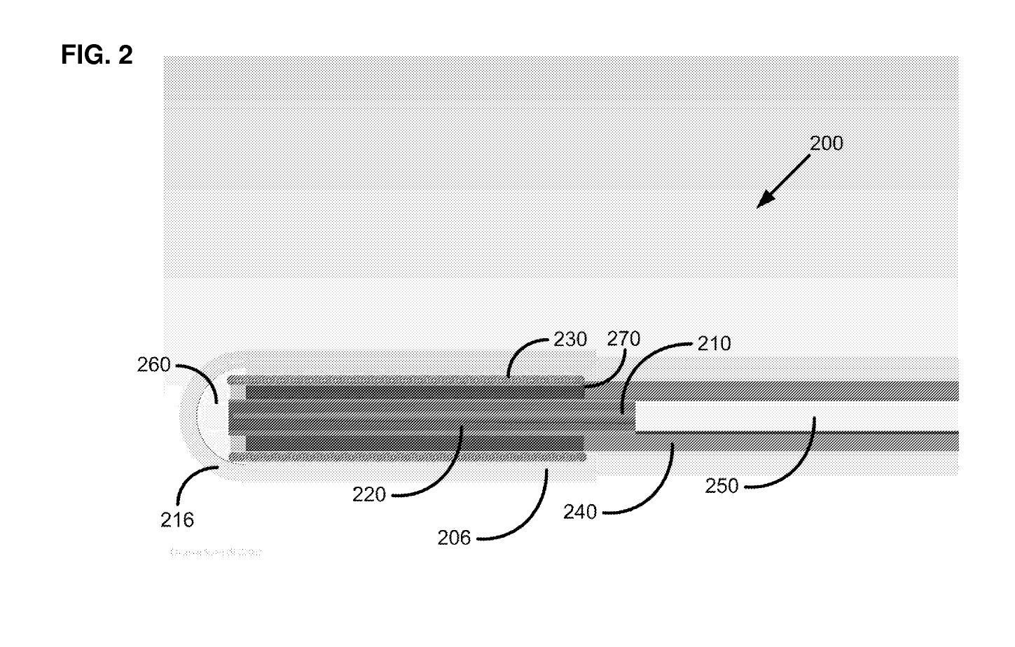

FIG. 2 shows another embodiment of an end of a guidewire 200. The guidewire 200 is covered in an outer insulation 206 for safe insertion into a human body. A rod 210 extends longitudinally along the entire length of the guidewire and can taper in diameter as it approaches a distal end 216. A first inner insulation layer 220 is adjacent the rod 210 and surrounds the rod so as to suppress receipt of MR signals. The first inner insulation layer 220 may only be present in the area of a helical coil 230 and the tapered rod allows for the additional insulation in this area. A second insulation layer 240 can extend along the entire length of the guidewire. The rod 210 can be positioned with a tube 250. The tube can be made of conductive, non-magnetic material, such a metal alloy of nickel and titanium (e.g., Nitinol). The rod 210 can also be made of Nitinol or similar conductive materials. The rod can be coated with more conductive metals, such as with gold. The solder coupling is shown at 260 and electrically connects the rod 210 with the helical coil 230. The rod 210, coil 230 and tube 250 together form a single antenna and a single channel that receives MR signals and transmits the MR signals to a signal processing system for analysis. A second solder joint 270 can also be present at the proximal end of the helical coil. Frequency and phase information can be detected and analyzed in order to determine a position of the received signal and project an anatomical background image on a display so that a shaft and tip of the guidewire can be seen.

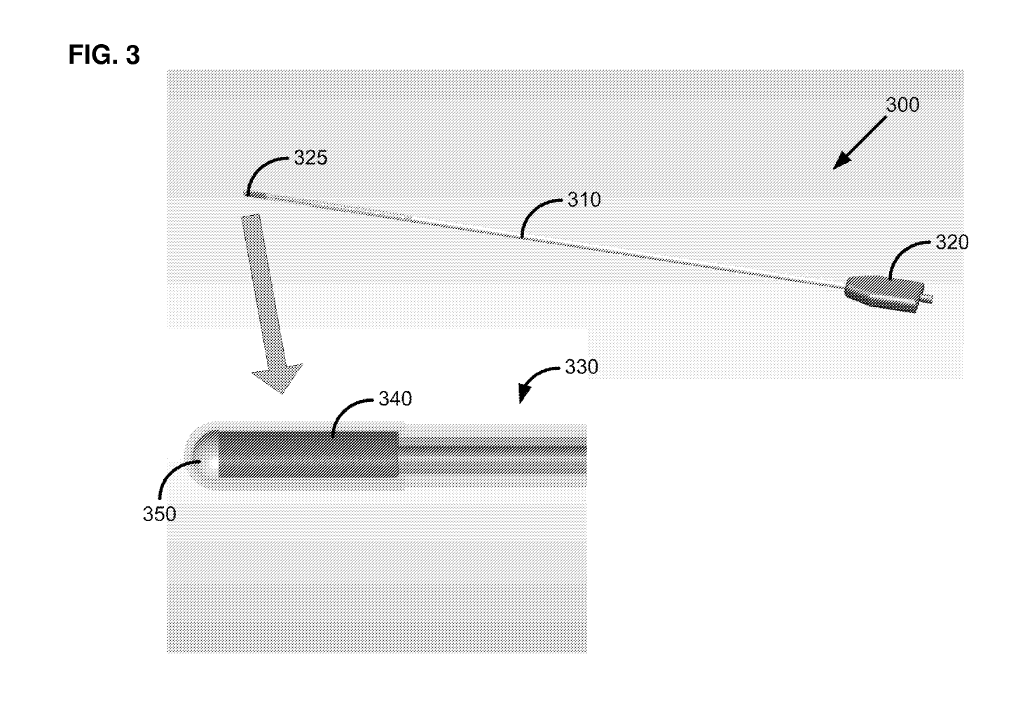

FIG. 3 shows a view of an embodiment of an entire guidewire 300. The shaft 310 can be any desired length and includes a connector 320 for attaching to a signal processing system discussed below. The distal end 325 of the guidewire can be any of the embodiments described herein and is shown generically at 330. The helical coil 340 and solder joint 350 are similar to those already described.

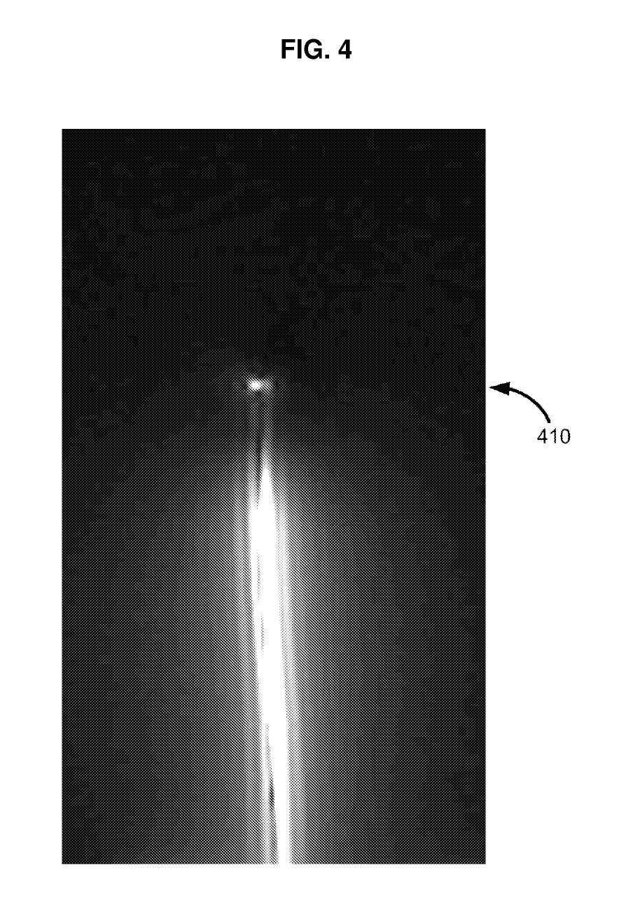

FIG. 4 shows a MRI image wherein a tip is shown as a dot centrally located and pointed to by arrow 410 is clearly visible and separated from a rest of the shaft. A suppressed region (seen as a dark space) between the tip and the shaft is due to the tightly wound helical coil and insulation within the coil.

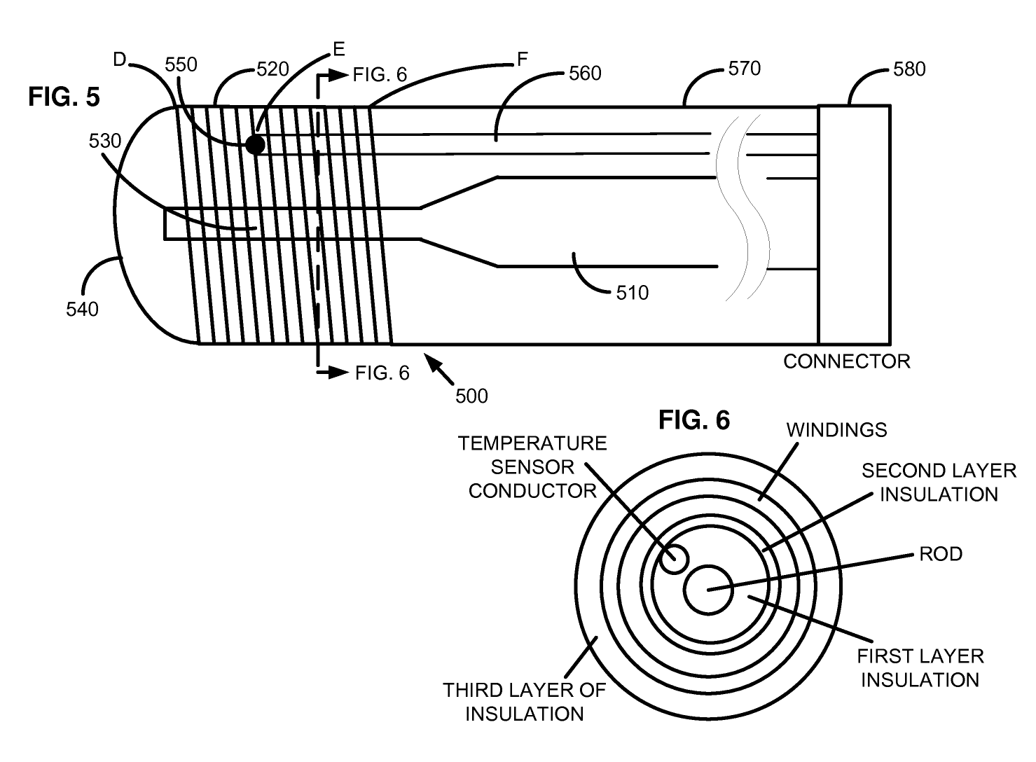

FIG. 5 shows another embodiment of a guidewire 500. A first portion of a rod 510 is not surrounded by the helical coil 520, while a second portion 530 is within the coil. The first portion of the rod 510 can have a larger diameter than the second portion 530, such that the rod tapers as it approaches the distal end 540 of the guidewire. The single channel guidewire includes an embedded port for a temperature sensor 550, such as a thermocouple, and a cable 560 (e.g., wire, fiber optic, etc.) attached thereto enclosed within an outer guidewire body 570. The temperature sensor is shown within the helical coil, but can be in any desired hot spot in which temperature information is desired. The guidewire can be any desired length and can be coupled via a connector 580 to a signal processing system, as is well understood in the art. Temperature information can be valuable to ensure that the guidewire does not exceed medical standards. Typical coil lengths can be around one inch in length. For longer coils (e.g., 2 inches), it was found that a small hole can be placed in the solder tip in order to lower permeability and increase magnetic field line density.

FIG. 6 shows a cross-sectional view along lines 6-6 of FIG. 5. As can be seen from the cross sectional view, the inner rod is centrally located and surrounded by a first insulation layer, which can be only in the area of the coil, a second insulation layer, which can extend the full length of the guidewire, and the helical coil, shown as an individual winding. The third insulation layer covers the entire guidewire to insulate the guidewire from body fluids.

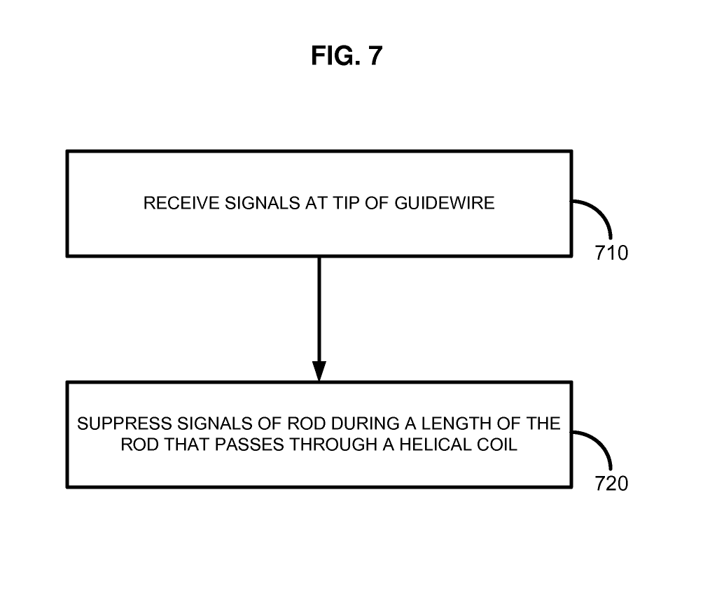

FIG. 7 is a flowchart of a method for viewing a conspicuous tip of a guidewire. In process block 710, the signals are received at the tip of the guidewire using the conductive joint. In process block 720, signals are suppressed over a length of a rod as it passes beneath a helical coil, while a remainder of the rod does receive signals. Thus, a gap is created between the tip and the shaft of the guidewire to allow easy visibility of both.

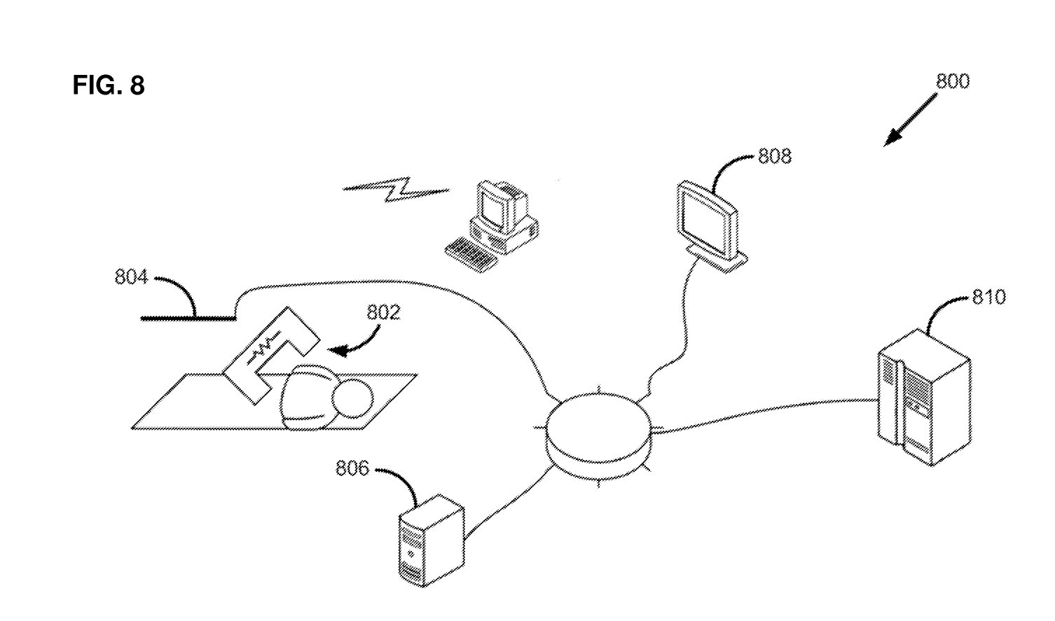

FIG. 8 illustrates a system 800 in which the guidewire described herein can be used. The MRI system can include an MRI scanner 802, an active guidewire 804, according to any of the embodiments described above, and signal processing system 806 electrically connected to the active guidewire through the single channel described above. A tuning circuit (not shown) can be coupled to the guidewire as is well understood in the art. The tuning circuit can be incorporated as part of the signal processing unit 806 or can be coupled to both the processing unit 806 and the guidewire 804. The guidewire 804 is constructed to provide signal information indicative of a shaft portion of the guidewire and a distal tip portion. The system 800 can include a display 808 for visualizing the guidewire similar to FIG. 4. Additional components 810 can be connected for storage, if desired.

In conventional conductive guidewires, there is a “hot spot” representing a portion of the device that reaches a greatest temperature located generally at the distal tip. In specific implementations as described herein, this “hot spot” is repositioned along the guidewire proximally of the distal tip. As a result, measuring a real time temperature increase from RF induced heating under MRI with a fiber optic temperature probe is easier. During typical use of a guidewire, the flexible distal tip is moved in ways such that it contacts surfaces (e.g., the surfaces of vessels, organs, etc.) frequently. If the hot spot is located at the distal tip, then the distal end of the fiber optic probe would need to be located at the distal tip. Typically, such a fiber optic has a GaAs crystal at its distal end, and this crystal would be subject to possible damage from the frequent contacts between the distal tip and adjacent surfaces. Further, a distal tip with an internal curvature might not allow the planar distal end of the probe to be placed as close to the distal tip as desired. Rather, it has been discovered that the hot spot can be positioned proximally of the distal tip, taking into account one or more of the following factors: the profile of the inner rod, the thickness of the insulation layer(s), the inner and outer diameters of the solenoid coil, the solenoid coil length and wire diameter, the solenoid coil insulation material(s), the soldering locations, etc., to achieve the desired results for different guidewire configurations.

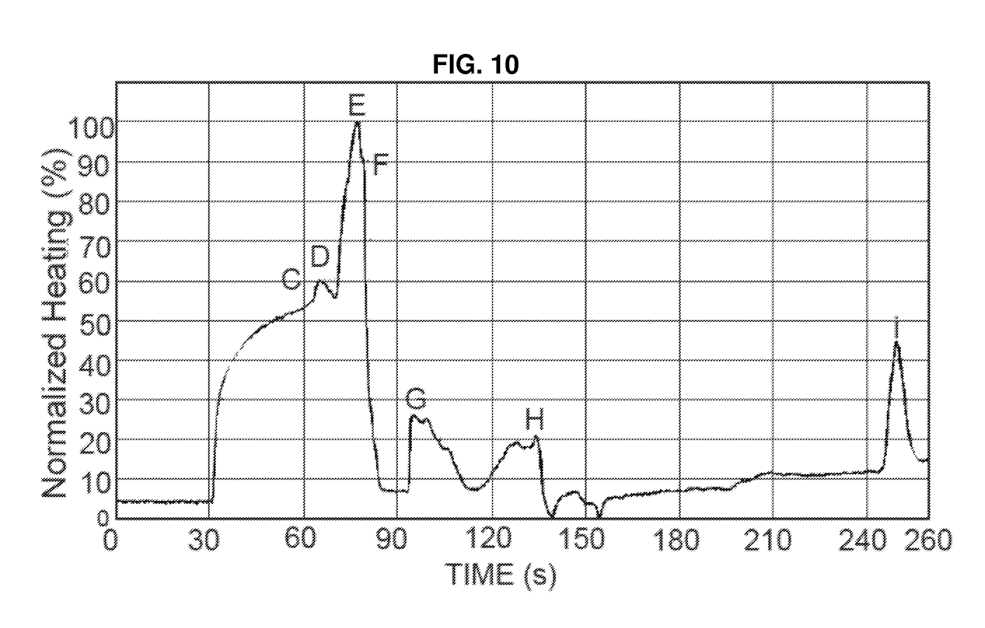

FIG. 10 is a graph or heating profile of normalized heating over time for a guidewire with a temperature probe that is subjected to heating while the temperature probe is slowly withdrawn in the proximal direction. Point D on the graph corresponds to the distal tip. As can be seen from the graph, the temperature at Point E (hottest spot), which is spaced proximally of the distal tip, has a higher temperature than Point D, and the highest temperature over the length of the guidewire. Referring to FIG. 5, the relative locations of Points D, E and F are shown. Point F is located at the proximal end of the coil 520. Points G (junction where inner corewire enters hypotube), H (guidewire entry point into gel) and I (MMCX connector), are not specifically shown in the drawings, but are generally located proximal of Point F. Points A, B and C, which are also not specifically shown in the drawings, are located distally of Point D.

As discussed above, in the various implementations, the coil is constructed to have a closed pitch configuration. Stated differently, the coil is constructed so that adjacent windings are not separated by gaps. As best shown in FIGS. 1, 2 and 5, the coils 120, 230, 520, respectively, are constructed such there are no gaps between adjacent windings. The closed pitch configuration of the coil helps to create a “null” zone in the received signal profile for the guidewire, i.e., because the side surface of the coil is substantially closed, the magnetic field is contained within the coil. If the windings are spaced from each other, then the magnetic field energy escapes and no null zone can be discerned.

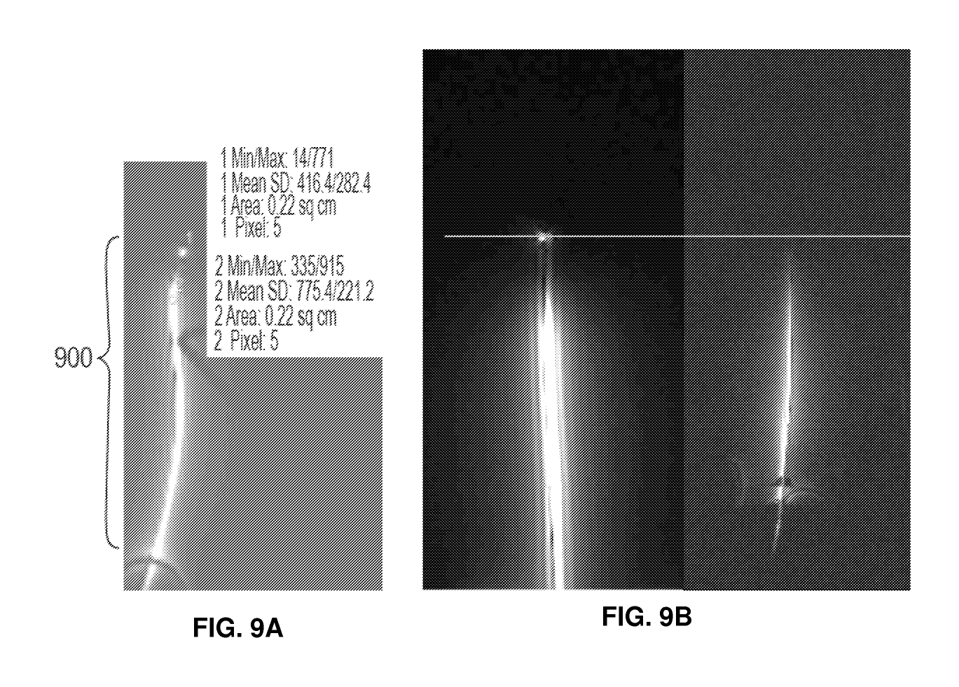

Use of a guidewire with a closed pitch coil and the resulting null zone produces a received signal profile that is unique and allows the operator to easily distinguish the shaft signal from the tip signal. Referring to FIG. 9B, the left side of the figure is an image produced using a guidewire with a closed pitch coil and producing a conspicuous tip signal (i.e., the spot of brightness at the location of the added horizontal line) and a distinct shaft signal spaced from the tip signal by the interspersed null zone. The right side of the figure shows the image produced by a conventional guidewire without a closed pitch coil, which was aligned to be at the same position as the guidewire on the left side. Although the shaft signal is visible, the tip signal, which should on the right side image appear in the area of the horizontal line is not readily discernible, or is at least not distinct from the tip signal.

The surface area of the solder and the ratio of the solenoid coil diameter to the inner rod diameter ratio are factors that affect resonant LC properties of the structure. In the described implementations, these properties are optimized for 0.035 in diameter guidewires, but the same principles can be applied to guidewires of different sizes and configurations.

In some applications, the distal portion of the guidewire is curved or “bent” rather than straight. For example, as shown in the image of FIG. 9A, the distal portion 900 curves to the left looking in the distal direction from the shaft to the distal end. The shaft signal is marked by the numeral “2” in the figure. The tip signal is marked by the numeral “1” in the figure. As noted above, it is important to use a closed pitch coil configuration that prevents gaps between adjacent windings. A standard closed pitch coil in a straight configuration will deform when installed in a curved distal portion, causing gaps to occur between windings on the long side of the curve, which would lead to a poor or nonexistent null zone.

It has been discovered that through careful measurement and forming techniques, a closed pitch coil suitable for a curved distal portion can be formed. The final curved geometry is carefully measured, and a metal mold for a coil corresponding to the final curved geometry is made. By forming the coil from a shape memory metal alloy such as nitinol, the coil can be molded to the correct final curved geometry, yet with the ability to deform during installation. As the coil is finally positioned, it will assume the proper final curved geometry and no gaps between the winding will be present. The windings can be coated with parylene or other insulating material with a high dielectric constant.

Referring again to FIG. 9A, it can be seen that the maximum signal strengths for the distal tip (771) and the shaft (915) are of the same magnitude, and the distal tip signal is desirably strong.

In described implementations, the guidewire has a dedicated port through which a fiber optic temperature probe or similar device) can be advanced and withdrawn along the guidewire shaft during a procedure. This is especially useful in conducting testing, such as RF safety, before clinical use. During such a test, the guidewire is arranged in a phantom and subjected to heating while the probe is withdrawn (a temperature probe pullback test). Areas of thinner insulation or other discontinuities, which might not be discovered through a visual inspection, create conspicuous hot spots that are easy to discern on a graph similar to FIG. 10.

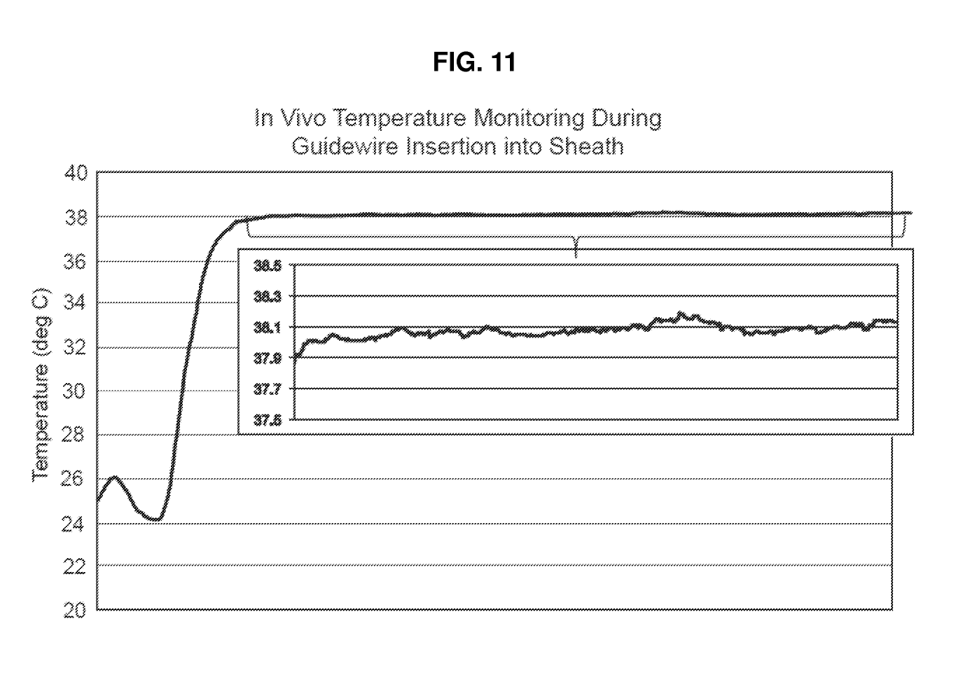

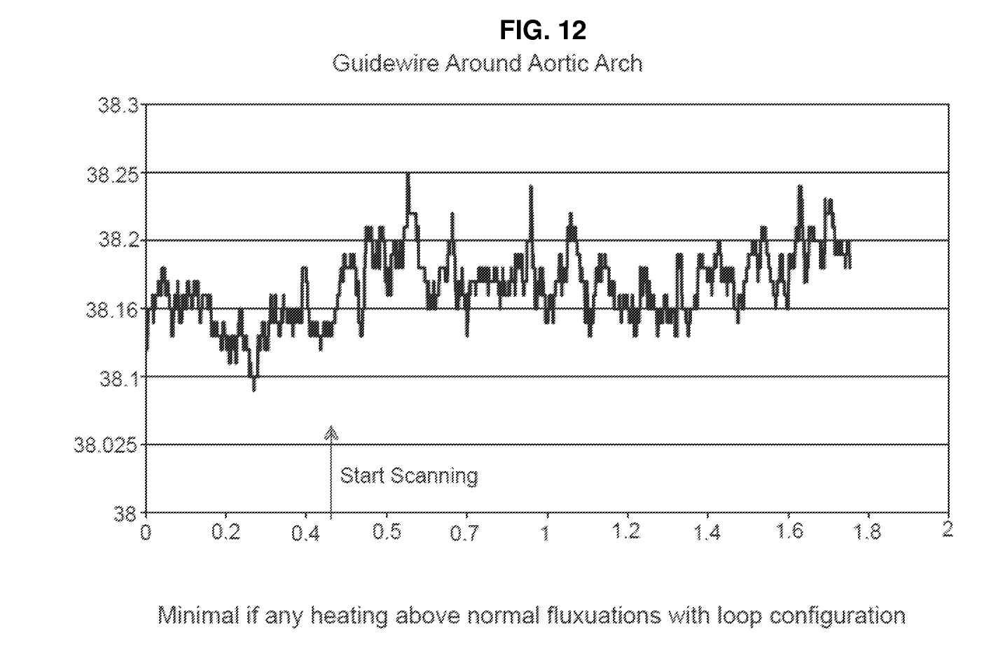

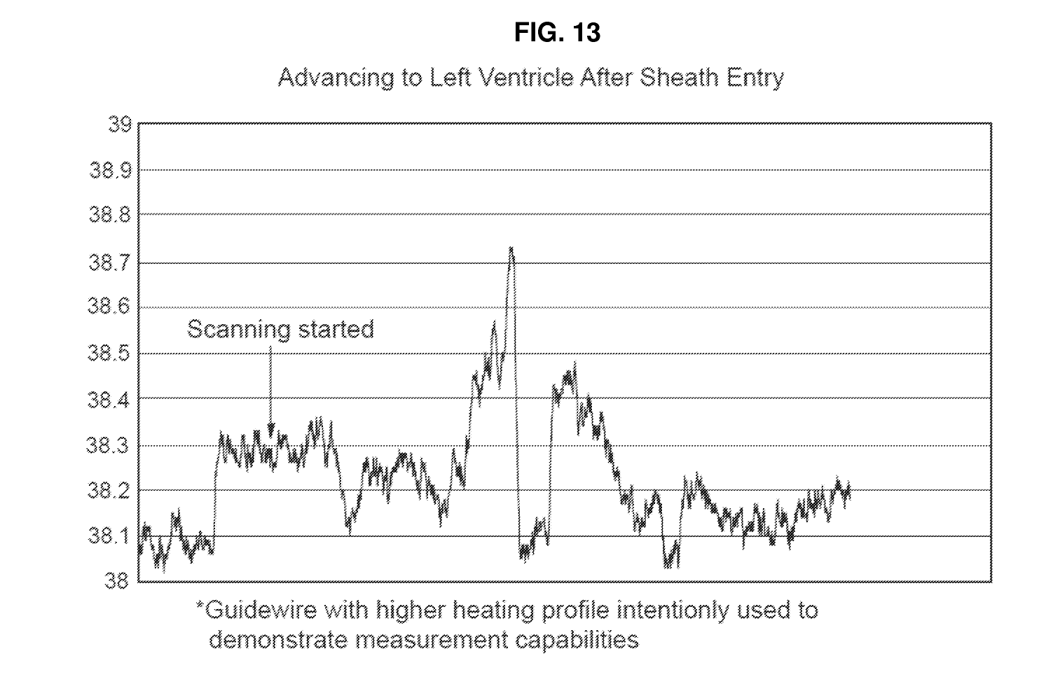

FIGS. 11-13 are temperature profile graphs for a described implementation of the guidewire at different stages in a procedure. As can be seen in FIG. 11, the measured temperature during insertion of the guidewire into a sheath rises rapidly and then reaches a highly uniform temperature (see also the inset magnified graph of FIG. 11). FIG. 12 shows the temperature profile after sheath entry and upon advancing to the left ventricle. As noted, a guidewire with a higher heating profile was used to demonstrate the measurement capabilities of the system. FIG. 13 shows the temperature profile for the guidewire in the area around the aortic arch. As noted, this profile shows that only fluctuations in the normal range occur during this phase of the procedure.

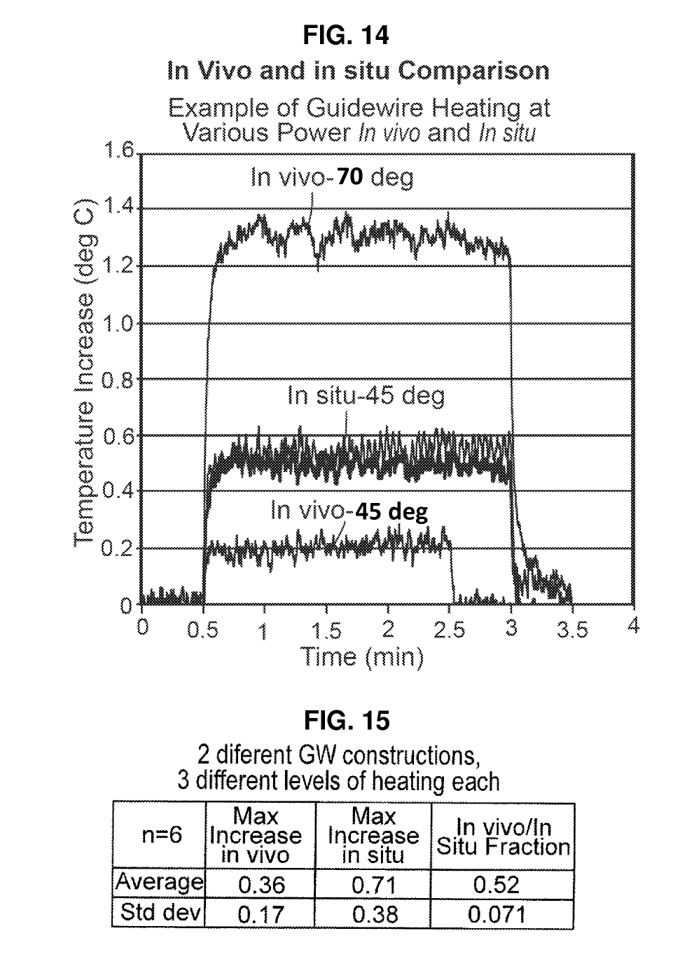

FIG. 14 is a comparison of three different guidewire heating conditions: (1) an in vivo condition at a 45 degree flip angle, (2) an in situ condition at a 45 degree flip angle, and (3) an in vivo condition at a 70 degree flip angle. FIG. 15 is a chart summarizing a statistical analysis of the three conditions. In general, the results depicted in the figures show that the described guidewire implementations have comparable or better performance than conventional guidewires.

In describing embodiments of the present invention illustrated in the drawings, specific terminology is employed for the sake of clarity. However, the invention is not intended to be limited to the specific terminology so selected. It is to be understood that each specific element includes all technical equivalents which operate in a similar manner to accomplish a similar purpose. The embodiments illustrated and discussed in this specification are intended only to teach those skilled in the art the best way known to the inventors at the time of filing to make and use the disclosed embodiments. The embodiments may be modified or varied, and elements added or omitted, without departing from the invention, as appreciated by those skilled in the art in light of the above teachings.

The disclosed methods, apparatus, and systems should not be construed as limiting in any way. Instead, the present disclosure is directed toward all novel and nonobvious features and aspects of the various disclosed embodiments, alone and in various combinations and subcombinations with one another. The disclosed methods, apparatus, and systems are not limited to any specific aspect or feature or combination thereof, nor do the disclosed embodiments require that any one or more specific advantages be present or problems be solved.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope of these claims.

We claim:

1. A guidewire for use with magnetic resonance imaging, comprising:
a guidewire body having a distal end and a proximal end;
an antenna disposed in the guidewire body, the antenna being formed from a rod passing through the guidewire body and a helical coil positioned at the distal end, the helical coil having multiple windings without a gap between the windings;
the rod passing through the windings of the helical coil and coupled to the helical coil using a conductive joint positioned at an end of the rod and at an end of the helical coil; and
insulation positioned between the rod and the windings of the helical coil.

2. The guidewire of claim 1, wherein the rod is formed from a non-magnetic material.

3. The guidewire of claim 1, wherein the conductive joint is an electrical connection.

4. The guidewire of claim 1, wherein the rod has a first diameter for a first portion of the rod, which is positioned outside of the windings of the helical coil, and a second diameter for a second portion of the rod that is positioned within the windings.

5. The guidewire of claim 1, wherein the conductive joint has a semispherical shape so as to maximize a conductive surface area.

6. The guidewire of claim 1, wherein the rod and the helical coil form a single channel that is electrically coupled at the proximal end, distal end or both ends of the helical coil to a signal processing system.

7. The guidewire of claim 1, wherein the helical coil, attached rod and an outer tube together form a dipole antenna.

8. The guidewire of claim 1, wherein the helical coil comprises a closed pitch solenoid coil.

9. The guidewire of claim 1, wherein the helical coil suppresses signals from being received from a portion of the rod within the windings.

10. The guidewire of claim 1, further comprising a temperature sensor in the guidewire to monitor in real-time the temperature of the distal end of the guidewire or any other hot spot.

11. The guidewire of claim 1, wherein the conductive joint forms a tip of the guidewire that receives signals for detection.

12. A method of visualizing a guidewire using magnetic resonance imaging, comprising:
receiving signals at a tip of a guidewire using a conductive joint which couples together a helical coil and a rod within the helical coil; and
suppressing signals received by the rod in an area where the rod passes through the helical coil so that the rod can be visualized as distinct from the tip.

13. The method of claim 12, wherein suppressing signals received by the rod is accomplished using the helical coil wherein windings of the helical coil are gapless.

14. The method of claim 13, wherein suppressing signals received by the rod is accomplished using insulation positioned between the rod and the windings.

15. The method of claim 13, further including forming a single channel using the helical coil, the conductive joint and the rod.

16. A guidewire for use with magnetic resonance imaging, comprising:
an antenna formed from a combination of a rod and a helical coil, with the rod extending through a center of the helical coil and being coupled thereto using a conductive joint at an end of the rod, the conductive joint forming a tip of the guidewire;
the rod having a first diameter and a second diameter, wherein the first diameter is along a length of the antenna prior to passing through the helical coil and the second diameter is where the rod passes through the helical coil; and
insulation surrounding the rod and positioned between the rod and the helical coil to suppress signals received by a portion of the rod having the second diameter.

17. The guidewire of claim 16, wherein the helical coil is tightly wound so that there are no gaps between windings to further suppress signals received by the rod along the portion of the rod having the second diameter.

18. The guidewire of claim 16, further comprising a temperature sensor positioned within the guidewire for sensing the temperature.

19. The guidewire of claim 16, further including a signal processing system coupled to the guidewire to identify the location of the tip as separate from the rod along a portion of the rod having the first diameter.

20. The guidewire of claim 16, further including a hole passing through the conductive joint for long coil applications.

21. A guidewire for use with magnetic resonance imaging, comprising:
an antenna formed from a combination of a rod and a helical coil, the coil defining an internal space and the rod being positioned to extend axially through the internal space and being coupled to the coil using a conductive joint at an end of the rod, the conductive joint forming a tip of the guidewire;
the guidewire having a null zone defined over an axial length between the conductive joint and a point proximal of the conductive joint, the null zone being operable to suppress signals received by a portion of the rod within the null zone, thereby producing a conspicuous distal tip signal.

22. The guidewire of claim 21, wherein the null zone produces a spatial separation between the distal tip signal and the shaft signal.

23. The guidewire of claim 21, wherein the coil has windings that are adjacent to each other over an axial distance corresponding to at least the length of the null zone.

24. The guidewire of claim 21, further comprising a temperature sensor positioned in and axially movable relative to the guidewire, the temperature sensor being configurable to monitor in real-time temperatures of interest along the guidewire.

25. The guidewire of claim 24, wherein the temperature sensor is configurable to measure for heating increases caused by defects in the guidewire.

26. The guidewire of claim 24, further comprising a dedicated port formed in the guidewire into which a distal end of the temperature sensor is inserted.

27. The guidewire of claim 21, wherein a distal end portion is curved and the helical coil has a corresponding preformed curved configuration without gaps between adjacent windings.

28. The guidewire of claim 27, wherein the helical coil is preformed of a shape memory alloy into the curved configuration.

29. The guidewire of claim 21, further comprising insulation in an annular region between the helical coil and the rod.

30. The guidewire of claim 21, further comprising multiple layers of insulation separating the rod from the coil.
